(19) United States
(12) Patent Application Publication
(10) Pub. No.: US 2026/0026708 A1
(43) Pub. Date: Jan. 29, 2026

(54) SYSTEMS AND METHODS FOR LEARNING POST-STROKE GAIT REHABILITATION STRATEGIES BY MODELING PATIENT-THERAPIST INTERACTION

(71) Applicants: Arizona Board of Regents on Behalf of Arizona State University, Tempe, AZ (US); Dignity Health, San Francisco, CA (US)

(72) Inventors: Seyed Mostafa Rezayat Sorkhabadi, Tempe, AZ (US); Wenlong Zhang, Chandler, AZ (US); Mason Smith, Chandler, AZ (US); Roozbeh Emami, Tempe, AZ (US); Trent Maruyama, Phoenix, AZ (US); Christina Kwasnica, San Francisco, CA (US); Melissa Fazekas, Phoenix, AZ (US); Rachel Lopez, Phoenix, AZ (US)

(21) Appl. No.: 18/786,007

(22) Filed: Jul. 26, 2024

(51) Int. Cl. A61B 5/103; A61B 5/00; G16H 20/30; G16H 40/67

(52) U.S. Cl. CPC A61B 5/1038; A61B 5/6807; A61B 5/7267; G16H 20/30; G16H 40/67; A61B 2505/09; A61B 2562/0219; A61B 2562/0247

(57) ABSTRACT

A system obtains high-resolution data of actual over-ground gait rehabilitation interactions through a custom-made wearable system for identifying abnormal gait patterns and the therapists' assistance strategies. The system implements an impedance learning algorithm with feature selection and a goal-directed attractor definition reproduces therapist assistance in a way that integrates clinical insights into the control of lower-limb exoskeletons.

### CROSS REFERENCE TO RELATED APPLICATIONS

This is a non-provisional application that claims benefit to U.S. Provisional Application Ser. No. 63/516,278, filed on Jul. 28, 2023, which is herein incorporated by reference in its entirety.

### GOVERNMENT SUPPORT

This invention was made with government support under 1756031, 1944833, and 1828010 awarded by the National Science Foundation. The government has certain rights in the invention.

### FIELD

The present disclosure generally relates to robotics-assisted rehabilitation, and in particular, to a system and associated method for modeling patient-therapist interaction for robotics-assisted gait rehabilitation.

### BACKGROUND

Each year, about 200,000 stroke survivors in the US are affected by lower-extremity hemiparesis. Common gait dysfunctions in post-stroke individuals involve a combination of impaired muscle strength, coordination, and proprioception. To help these patients regain their normal walking ability, manual gait therapy is currently provided by a physical therapist (PT) using motor learning principles. This one-to-one physical therapy is effective but time-consuming, costly, and labor-intensive.

It is with these observations in mind, among others, that various aspects of the present disclosure were conceived and developed.

### BRIEF DESCRIPTION OF THE DRAWINGS

FIGS. 1A and 1B are a pair of diagrams showing a robot-aided gait rehabilitation system outlined herein;

FIG. 2 is a diagram showing wearable sensors of the system of FIGS. 1A and 1B during a gait therapy session;

FIG. 3 is a diagram showing interpretation of relevant kinematics variables for a knee as discussed herein with respect to the system of FIGS. 1A and 1B;

FIGS. 4A and 4B are a pair of graphical representations showing visualization of collected data for two patients for development of the system of FIGS. 1A and 1B;

FIGS. 5A and 5B are a series of graphical representations showing gait pattern classification for knee flexion during a training session, an observed torque from a physical therapist, and assistance torque reproduced by a virtual impedance model of the system of FIGS. 1A and 1B;

FIG. 6 is a simplified diagram showing an example computing system for implementation of the system of FIGS. 1A and 1B; and

FIGS. 7A and 7B are process flow diagrams showing a method for implementation of the system of FIGS. 1A and 1B.

Corresponding reference characters indicate corresponding elements among the view of the drawings. The headings used in the figures do not limit the scope of the claims.

### SUMMARY

A method outlined herein includes: accessing, by a processor in communication with a memory, a set of observation signals for a time step, the set of observation signals being measured at a body part of a patient during a gait rehabilitation exercise; and determining, for a time step and by a prediction model implemented at the processor, a predicted attractor state and an assistive torque value for the time step based on the set of observation signals.

The set of observation signals can be measured by a plurality of sensors during the gait rehabilitation exercise, the plurality of sensors including: one or more pressure sensor arrays associated with the body part and being operable for measuring components of an assistive force value and a ground reaction force associated with the body part during the gait rehabilitation exercise; and an inertial measurement sensor associated with the body part and being operable for measuring one or more kinematic values associated with the body part including a shank displacement angle and a thigh displacement angle.

The method can further include determining, at the processor and based on the predicted attractor state and the assistive torque value for the time step, one or more actuation inputs for application to an exoskeleton actuation system positioned along the body part of the patient during a gait rehabilitation exercise.

For determining the predicted attractor state and the assistive torque value, the method can further include: determining a set of observation values based on the set of observation signals, the set of observation values including: a state of a virtual impedance model that includes one or more observed angles and an observed weight-shift associated with the body part of the patient for the time step; and a time-varying stiffness value and a time-varying damping value of the virtual impedance model; the virtual impedance model relating the state and the time-varying stiffness value and the time-varying damping value to the assistive torque value and the predicted attractor state; and determining, at the processor and by a Gaussian Mixture Regression process, a probability that the set of observation values associated with the body part of the patient for the time step belongs to a gait pattern class characterized by a Gaussian Mixture Model, the gait pattern class being one of a plurality of gait pattern classes identified during parameterization of the Gaussian Mixture Model.

The set of observation values can further include: a state of a virtual impedance model that includes one or more observed angles and an observed weight-shift associated with the body part of the patient for a first previous time step; and a state of a virtual impedance model that includes one or more observed angles and an observed weight-shift associated with the body part of the patient for a second previous time step; where the time step and the first previous time step are spaced equally apart from one another and where the first previous time step and the second previous time step are spaced equally apart from one another.

The method can further include: determining, at the processor and based on the probability, the predicted attractor state and a residual torque value associated with the set of observation values for the time step; and determining, at the processor, the assistive torque value for the time step based on the predicted attractor state, the residual torque value, the state of the virtual impedance model, the time-varying stiffness value and the time-varying damping value.

For training the prediction model, the method can include: parameterizing the Gaussian Mixture Model to classify sets of observation data into a gait pattern class of the plurality of gait pattern classes using a set of training data, each instance of the set of training data including: a state of a virtual impedance model for a first time step; a state of the virtual impedance model for a second time step; a state of the virtual impedance model for a third time step; and an attractor state of the virtual impedance model associated with the third time step; where the first time step and the second time step are spaced equally apart from one another and where the second step and the third time step are spaced equally apart from one another. Further, the method can include: estimating, at the processor and using a posterior probability associated with the Gaussian Mixture Model, a class stiffness gain value, a class damping gain value, and a class mean residual torque for each gait pattern class of the plurality of gait pattern classes.

A system includes: a wearable sensor system having a plurality of sensors that obtain a set of observation signals during a gait rehabilitation exercise, the set of observation signals including: a ground reaction force associated with a body part of a patient; and one or more kinematic values associated with the body part including a shank displacement angle and a thigh displacement angle; and a computing device in communication with the wearable sensor system, the computing device including a processor in communication with a memory, the memory including instructions executable by the processor to determine, for a time step and by a prediction model implemented at the processor, a predicted attractor state and an assistive torque value for the time step based on the set of observation signals.

The system can further include an exoskeleton actuation system positioned along the body part and in communication with the computing device, the memory of the computing device further including instructions executable by the processor to: determine, at the processor and based on the predicted attractor state and the assistive torque value for the time step, one or more actuation inputs for application to the exoskeleton actuation system positioned along the body part of the patient during a gait rehabilitation exercise.

The plurality of sensors can include a first inertial measurement unit positioned along an upper brace of the wearable sensor system, the upper brace being positionable along a thigh of the patient, the first inertial measurement unit being operable for measuring one or more kinematic values associated with the body part including a thigh displacement angle; a second inertial measurement unit positioned along a lower brace of the wearable sensor system, the upper brace being positionable along a shank of the patient, the second inertial measurement unit being operable for measuring one or more kinematic values associated with the body part including a shank displacement angle; and a pressure sensor array positioned along a shoe of the wearable sensor system and being operable for measuring a ground reaction force associated with the body part, the shoe being positionable along a foot of the patient. Further, the plurality of sensors can include one or more pressure sensor arrays positioned along the upper brace and the lower brace of the wearable sensor system that are collectively operable for measuring an assistive force applied by a practitioner during the gait rehabilitation exercise.

In a further aspect, a non-transitory computer readable medium includes instructions executable by a processor to: access a set of observation signals for a time step, the set of observation signals being measured at a body part of a patient during a gait rehabilitation exercise; and determine, for a time step and by a prediction model implemented at the processor, a predicted attractor state and an assistive torque value for the time step based on the set of observation signals.

### DETAILED DESCRIPTION

For safe and effective robot-aided gait rehabilitation, it is essential to incorporate the knowledge and expertise of physical therapists. Toward this goal, systems outlined herein directly learn from physical therapists' demonstrations of manual gait assistance in stroke rehabilitation. Lower-limb kinematics of patients and assistive force applied by therapists to the patient's leg are measured using a wearable sensing system which includes a custom-made force sensing array. The collected data is then used to characterize a therapist's strategies in response to unique gait behaviors found within a patient's gait. Preliminary analysis shows that knee extension and weight-shifting are the most important features that shape a therapist's assistance strategies. These key features are then integrated into a virtual impedance model to predict the therapist's assistive torque. This model benefits from a goal-directed attractor and representative features that allow intuitive characterization and estimation of a therapist's assistance strategies. The resulting model is able to accurately capture high-level therapist behaviors over the course of a full training session (r2=0.92, RMSE=0.23 Nm) while still explaining some of the more nuanced behaviors contained in individual strides (r2=0.53, RMSE=0.61 Nm). This work provides a new approach to control wearable robotics in the sense of directly encoding the decision-making process of physical therapists into a safe human-robot interaction framework for gait rehabilitation.

### I. Introduction

To make gait therapy easily accessible and affordable for patients, lower-limb exoskeletons have been studied for post-stroke gait rehabilitation. Despite considerable progress in wearable exoskeletons, a major challenge remains in designing personalized robot control strategies for different patients. Previous studies have suggested that modeling and identifying human-human sensorimotor interactions can lead to the development of robots that physically interact and move with humans in an intuitive and efficient manner.

Physical rehabilitation is a form of human-human interaction in which the goal of the PT is to train patients to improve their motor performance. However, in the context of rehabilitation robotics, there is no widely accepted framework to learn from this human-human interaction. This is due to a scarcity of studies that collect and model the interactions between PTs and patients to help identify the sensorimotor principles of such interactions.

### 1-A. Previous Studies

One study collected the interaction force and leg kinematics in treadmill-based training and showed that different PTs applied significantly different forces, resulting in different leg kinematics. However, no specific strategy on how the forces contributed to the task performance or modeling of the assistance was provided. In one study, a backdrivable robot was used to measure foot kinematics and assist in manual stepping training of spinal cord injury patients. The robot's linear actuators were connected to the treadmill sidebars, and the apex of the robot was attached to the subject's ankle joint center through two linkages and a revolute joint, applying a 2-D force directly to the foot. The measured average trajectory of the apex in manual training was used as the desired trajectory of an adaptive impedance controller of the robot. In that study, interaction forces and joint trajectories were not collected and the impedance gains were tuned on a step-by-step basis with an error-based learning law error. Therefore, it fails to capture the variation in the imposed trajectories by the PTs as well as the impedance gains within a gait cycle (different gait phases), which can lead to poor generalizability. In a more recent study, impedance-based learning was achieved from demonstration (LfD) framework targeting foot-dropping assistance during treadmill-based gait therapy, using a robotic arm. Despite the successful modeling and implementation, the experiments did not include the actual physical therapist-patient (PT-P) interaction to extract gait rehabilitation insights.

### 1.B. Overview

The present disclosure outlines a custom-made wearable sensor system that measures interaction forces and leg kinematics during physical therapy, particularly for post-stroke gait rehabilitation of hemiparetic patients with moderate weakness in their quadriceps muscles. The collected data is then used to characterize the patient's gait patterns and the corresponding assistive torques applied by a physical therapist (PT). The results offer important insights into how gait rehabilitation principles, when used in tandem with clinical experience and patient gait observations, can lead to better timing and magnitude of assistance. To achieve this, an LfD-based approach to virtual impedance control is implemented as the mechanism for reproducing PT assistance and incorporating these clinical insights.

Impedance controllers are a second-order dynamic model inspired by biological mechanisms for compliant interaction, and can be widely used for controlling robots that physically interact with humans due to its admittance of and stability in response to disturbances, which keeps interaction forces within a safe range. In robot-aided rehabilitation contexts, impedance control also provides assist-as-needed interaction which has been shown to promote motor learning and recovery. Additionally, impedance control admits disturbances (e.g., human interaction forces such as those applied by the physical therapist and/or the patient) instead of rejecting them, as in simpler direct position and velocity controllers, which can lead to discomfort and safety concerns in rehabilitation. Conversely, complex approaches for learning predictive torque control models (e.g., inverse reinforcement learning (IRL) and artificial neural network (ANN)) may suffer from issues of tractability, non-uniqueness, poor interpretability, or large amounts of data required for training. The latter issue is particularly relevant in rehabilitation settings since access to patients for training is often limited. Furthermore, learning impedance strategies over patient states enables learning an interpretable model with a physical intuition due to correlation with a spring-mass damper system. Consequently, impedance control provides a means for model verification and extraction of PT assistance insights in a way not immediately available in other approaches. Therefore, impedance control affords modeling PT strategies while providing the aforementioned benefits for complexity, interpretability, physical intuition, safety, and accuracy of the learned model.

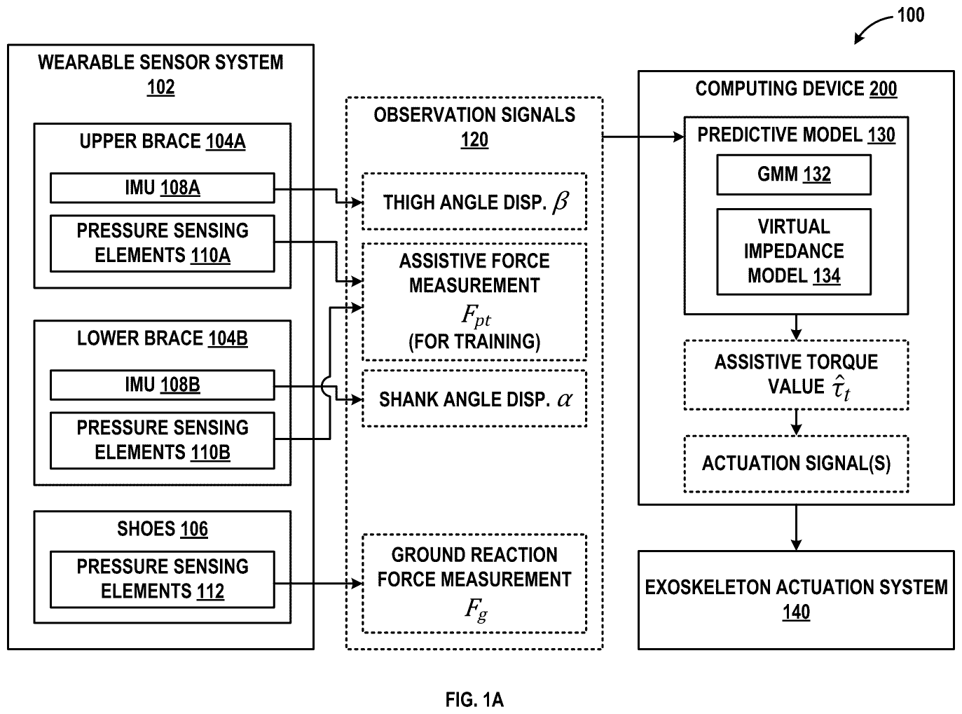

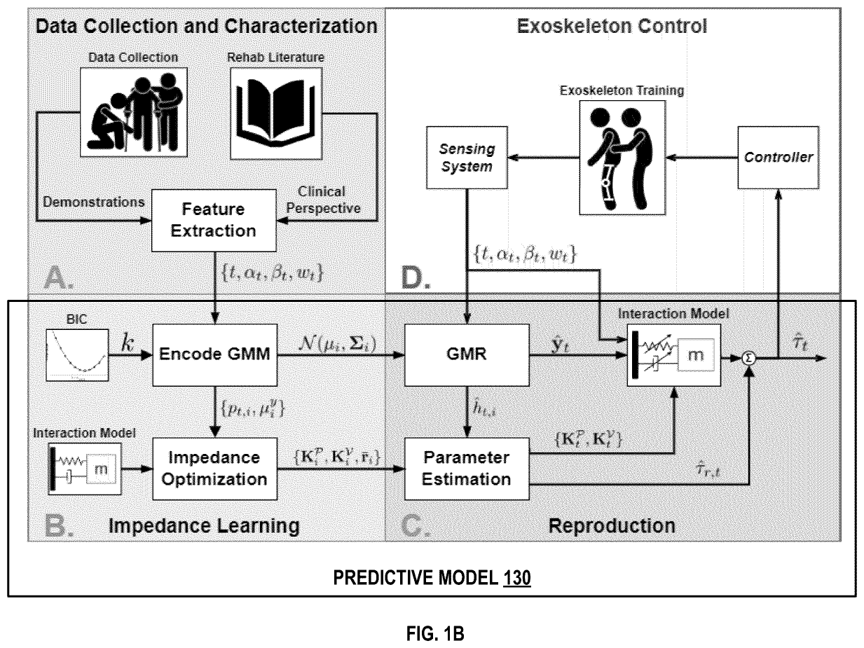

The contributions of this disclosure are summarized as shown in FIGS. 1A and 1B which illustrate a robot-aided gait rehabilitation framework (“system 100”). FIG. 1A is a simplified block diagram showing an implementation of the system 100, and FIG. 1B is a diagram showing functionality of the system 100 shown in FIG. 1A.

As shown in FIG. 1A, the system 100 can include a wearable sensor system 102 which obtains observation signals 120 during gait rehabilitation exercises for a patient, and a computing device 200 in communication with the wearable sensor system 102 that obtains or otherwise predicts an assistive torque value \hat{τ}ₜ that would be applied by a PT during gait rehabilitation using a predictive model 130. The assistive torque value \hat{τ}ₜ can be used to generate actuation signals for application as input to an exoskeleton actuation system 140 which may be worn by the patient during a gait rehabilitation exercise, and may apply an assistive torque according to the assistive torque value \hat{τ}ₜ.

The wearable sensor system 102 can include an upper brace 104A which can be positioned along a thigh of the patient, a lower brace 104B which can be positioned along a shank of the patient, and “smart shoes” (shoes 106) which collectively measure observation signals 120. The upper brace 104A can include an inertial measurement unit (IMU) 108A which can measure a thigh angle displacement β, and the lower brace 104B can include an inertial measurement unit (IMU) 108B which can measure a shank angle displacement α. Further, the upper brace 104A can include pressure sensing elements 110A and the lower brace 104B can include pressure sensing elements 110B that collectively measure an assistive force measurement Fₚₜ applied by the PT during gait rehabilitation. This assistive force measurement Fₚₜ can be used to train the predictive model 130 to predict the assistive torque value \hat{τ}ₜ that should be applied to the patient based on observation signals 120 as outlined further herein. Further, the shoes 106 can include pressure sensing elements 112 that measure a ground reaction force measurement F_{g}, which may be used to obtain a weight shift value w that can be used along with the shank angle displacement α and the thigh angle displacement β as predictors for the assistive torque value \hat{τ}ₜ.

The predictive model 130 can incorporate a Gaussian Mixture Model (GMM) 132 which can be used with a Gaussian Mixture Regression (GMR) algorithm to identify, for each respective gait pattern class of a plurality of gait pattern classes, a probability ĥ_{t,i} that the set of observation signals belongs to the gait pattern class iε{1, 2, . . . , k}. The predictive model 130 can also include a virtual impedance model 134 (Eqs. (1), (3)) that correlates a current state xₜ associated with the observation signals, a predicted attractor state ŷₜ, a time-varying stiffness value [symbol] , and a time-varying damping value

$${K}_{t}^{𝒱}$$

with the assistive torque value \hat{τ}ₜ. Further, as discussed herein with respect to Eq. (3), the virtual impedance model 134 can further incorporate a residual torque value rₜ which can represent additional or otherwise unexplained forces that a PT may apply, such as preemptively applying a stabilizing force in case of knee instability.

As shown in FIG. 1B, during a training phase for the predictive model 130, the observation signals 120 can incorporate measured assistive forces that are applied by a PT. First, for training, the system 100 obtains high-resolution data of actual over-ground gait rehabilitation interactions through a wearable sensor system 102 which can be worn by the patient during data collection and active assistance. The high-resolution data can be used for identifying abnormal gait patterns of the patient as well as for characterizing assistance strategies employed by the PT. Further, the system 100 applies an impedance learning algorithm with insightful feature selection and a goal-directed attractor definition. The impedance learning algorithm provides a means to reproduce PT assistance in a way that integrates clinical insights into the control of lower-limb exoskeletons. It is believed that no other work has collected such data in this setting to model PT decision-making for the reproduction of assistive torque in gait rehabilitation.

FIG. 1B shows an overview of the robot-aided gait rehabilitation framework (the system 100) presented in this disclosure. In panel (A) of FIG. 1B, data is collected from gait rehabilitation sessions and analyzed using insights from the clinical literature. This analysis informs a Gaussian mixture model (GMM) inspired impedance learning algorithm shown in panel (B) of FIG. 1B that is used to reproduce observed PT torque behaviors by applying a Gaussian mixture regression (GMR) prediction algorithm shown in panel (C) of FIG. 1B. These reproduced torques are evaluated on a subset of patient states to test the ability of the system to replicate PT behaviors when applied in the lower-limb exoskeleton during future work as shown in panel (D) of FIG. 1B.

### II. Methods

### A. Data Collection System

To capture the interaction dynamics between the PT and the patient, the wearable sensor system 102 records forces exerted by the PT, knee joint kinematics, and ground reaction forces at the affected side.

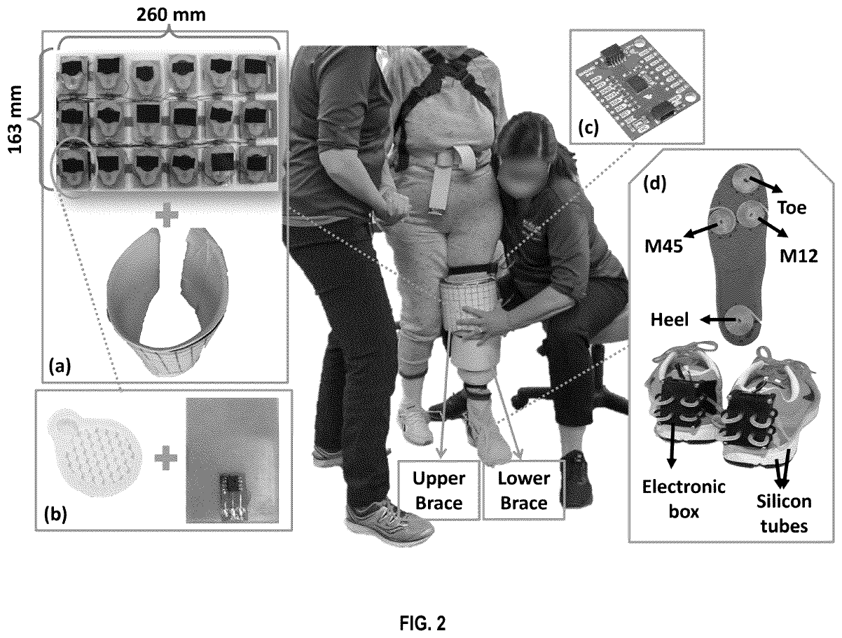

During the gait therapy sessions, the PT contacts and exerts force on different areas of the body. However, to stabilize the knee motion during weight-shifting and prevent knee buckling, the assistive forces appear to be mostly focused on two areas: the upper and lower anterior knee (where the force sensors are placed as shown in FIG. 2). Therefore, two separate soft force sensor braces are presented for each segment (e.g., the upper brace 104A for the upper anterior knee and the lower brace 104B for the lower anterior knee), intending to capture both the magnitude and distribution of the force in a non-intrusive manner. The distribution of the force is necessary to calculate the aggregate assistive torque. Each brace embodies a flexible pressure sensor array (3×6 and 2×5 for the upper brace 104A and lower brace 104B, respectively) including air pressure sensors enclosed by air-filled silicon pads with an elastomeric pillar array (see panel (B) of FIG. 2). The force is measured by the increase in the air pressure caused by pillar compression. This design allows for a compact structure by introducing a high measured force-to-volume ratio and is shown to have a linear and repeatable pressure-force behavior. Each sensor is calibrated separately with a linear model between measured force (N) and sensor output (volts).

FIG. 2 shows an example implementation of the wearable sensor system 102 during a gait therapy session. Panel (A) of FIG. 2 shows components of the soft force sensor braces (e.g., the upper brace 104A and the lower brace 104B), including an array of pressure sensing elements held by a flexible 3D printed thermoplastic polyurethane (TPU) part (top of panel A), connected to the soft curved outer shell (bottom), which could easily fit a patient leg with different sizes (the two sensor braces weigh about 900 g in total). Panel (B) of FIG. 2 shows components of the pressure sensing unit, air-filled silicon pad (left), enclosing the absolute pressure sensor (right). As shown in panel (C) of FIG. 2, IMU sensors are placed at the shank and thigh. Panel (D) of FIG. 2 shows “Smart Shoes” for measuring ground reaction forces which include coiled silicon tubes placed at four points of contact in the insole (top of panel (D)) and the electronic box including the pressure sensors, microcontroller, and a wireless connectivity module (e.g., a WiFi module) (bottom of panel (D)).

Knee joint kinematic data were captured using inertial measurement sensors (in one example implementation, an off-the-shelf BNO085 IMU sensor, SparkFun Electronics, CO). The Smart Shoes (shoes 106) were used to capture ground reaction force (GRF) data of the affected side in real-time (FIG. 2). All data was collected by a host microcontroller (e.g., an Intel UP-Board, Intel Corp., CA), which along with a 12V battery and PCB boards was placed inside a backpack worn by the patient. The backpack weighed less than 500 g.

### B. Experimental Protocol

1) Patient Recruitment: Post-stroke patients with hemi-paretic gait, specifically with weakness in the quadriceps leading to knee instability, were recruited at Barrow Neurological Institute (BNI) in Phoenix, Arizona. Participants had to be able to ambulate with minimal assistance or less for up to 5 minutes with or without the use of a single-point cane and/or ankle foot orthosis (AFO). Participants with manual muscle testing (MMT) scores of knee flexion/extension less than 2+ were excluded from the study. Participants with modified Ashworth of hemiparetic lower extremity less than or equal to 1+, and with flexion contracture that cannot extend the knee beyond 10° were also excluded from the study. These criteria was set to allow for recruiting patients with less impairment, so the PTs' assistance would focus on correcting certain gait abnormalities related to knee instability. Before enrollment, a series of physical assessments were performed by the PTs to ensure that all the criteria were met. The protocol of the study was reviewed and approved by the institutional review board at BNI (protocol number 19-500-271-70-19).

Initially seven patients were recruited, out of which four patients' data are used in this study. Data from two patients were discarded as the PT assistance was not significant due to their relatively functional and independent gait. Thus the inclusion/exclusion criteria was adjusted accordingly to recruit patients with higher impairment levels. Another patient's data collection was unsuccessful because of technical difficulties with the wearable sensor system during the gait rehabilitation session. Anthropomorphic information and the knee and hip flexion/extension MMT scores of the four final patients are given in Table I.

TABLE I
Patient Anthropometric and MMT Data

Patient ID (P)

| Patient Information | 3 | 5 | 6 | 7 |
|---|---|---|---|---|
| MW Group | HMW | LMW | HMW | LMW |
| Anthropometry | | | | |
| Gender | F | F | F | F |
| Age | 27 | 44 | 68 | 28 |
| Weight (kg) | 64 | 76 | 83 | 53 |
| Height (cm) | 160 | 170 | 150 | 160 |
| Paretic Side | R | L | L | R |
| MMT | | | | |
| Knee Flexion | 2+ | 4− | 3+ | 4+ |
| Knee Extension | 4 | 4− | 4− | 4 |
| Hip Flexion | 4 | 4− | 3 | 4 |
| Hip Extension | 2+ | 4 | 3+ | 4 |

2) Experimental Procedure: The BNI team conducted participant enrollment after which a two-hour data collection session was scheduled with participants that consented. Each data collection session included three sub-sessions: 1) no training (NT), 2) training by the first PT (PT1), and 3) training by the second PT (PT2). Two PTs' data was collected in each session to study the similarity and differences in their gait rehabilitation strategies.

At the beginning of each session, patients first put on the wearable sensors with the help of the PTs. If the patient wore an AFO, it was removed and replaced by an ace wrap to prevent foot drop. In the first NT sub-session, the patients walked by themselves for three minutes, with minimal assistance from the PT only when needed for safety. Minimal assistance in this context is defined as assisting with less than 25% of the weight support and balancing, through pelvis and upper-body assistance. After the first sub-session was completed, the patient rested for at least five minutes depending on patient readiness. Next, the training with the PT1 began, in which the PT, sitting on a wheeled stool, facilitated the upper knee motion of the paretic side to support weight-shifting and knee stability. These two sub-sessions also lasted for three minutes each and included multiple 10-15 meter laps, at the end of which each patient would turn, and stay steady and straight for 10 seconds (to help with initializing IMUs and the force sensor). Real-time feedback for the quality of each gait cycle from PTs was collected (see point (5) of Sec. III-A below). After resting for at least five minutes, the other training sub-session was performed by PT2, similar to the previous sub-session. To assess if the data collection system interfered with patient kinematics or PT assistance, which would invalidate the data, both the patient and PT filled out a survey expressing their opinion on the training session and data collection system.

### III. Biomechanical Characterization

In this section, using the collected data, including the forms and recorded videos, quantitative and qualitative analyses are performed to characterize the patient's gait, along with facilitation strategies used by the PTs.

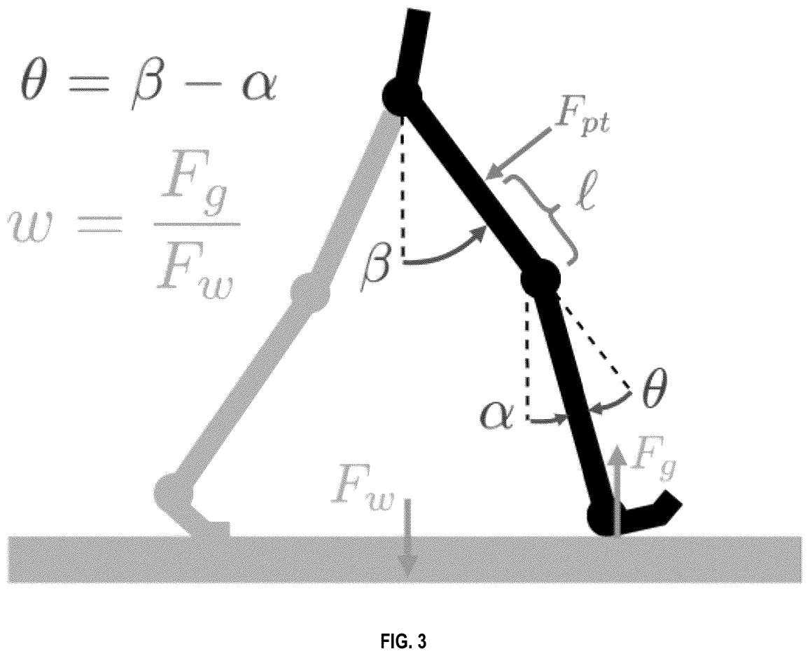

A kinematics diagram in FIG. 3 shows the relevant kinematic variables for knee angle displacement θ, shank angle displacement α, and (sagittal) thigh angle displacement β. It also shows a sagittal force Fₚₜ applied by the therapist. The ground reaction force F_{g} is applied at the foot (paretic side) and is used to calculate percent weight-shift w by scaling the ground reaction force F_{g} by the patient's weight F_{w}.

### A. Initial Data Processing

Initially, all sensor data was collected on a host PC at different frequencies. In the post-processing, all data were synchronized using the common time frame in the host PC and re-sampled to 75 Hz. The following variables were extracted:

1) Gait Sub-Phases and Percentage: Using the four readings from the Smart Shoe, it is possible to segment the gait into six sub-phases: heel-strike (HS), loading-response (LR), mid-stance (MST), terminal-stance (TST) or heel-rise, pre-swing (PSW) or toe-off, and swing (SW). The first five sub-phases (HS, LR, MST, TST, and PSW) belong to a “stance” phase. This segmentation is based on insole sensor measurements which can be classified using a fuzzy logic algorithm. By incorporating these sub-phases, the data can be segmented into individual gait cycles, and the stance time and stance phase percentage of each gait cycle can be extracted.

2) Lateral Weight-Shift: The sum of sensor readings obtained from the Smart Shoes gives the net force applied to the foot by the ground F_{g}. Normalizing this value by the weight of each patient F_{w}, the approximate percent of weight shifted on the paretic side w can be calculated at each time step.

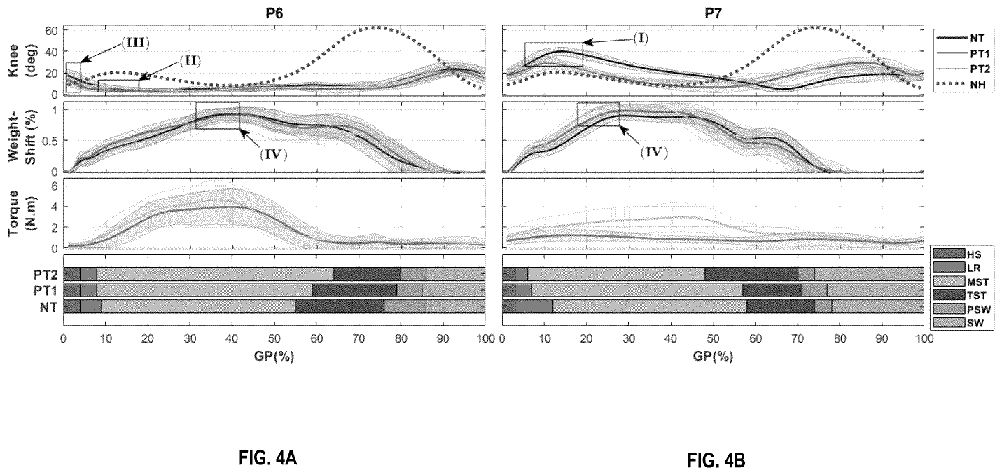

3) Joint Kinematics: The thigh and shank kinematics are calculated using the net quaternion rotation Δq=qq₀⁻¹ for each IMU where q is the current quaternion value and q₀ is the reference rotation established by having the PT place the patient's knee at full extension at the beginning of each lap. It was observed that a consistent reference q₀ was challenging to achieve and is estimated to produce an error of ±5°. This may explain the lack of knee hyperextension in the observed joint trajectories as seen in FIGS. 4A and 4B. Sagittal thigh angle displacement β and shank angle displacement α are then recovered by applying Δq to a unit vector and projecting it onto the sagittal plane. With this information, the patient's knee angle displacement θ can then be calculated as the difference between thigh angle displacement β and shank angle displacement α as seen in FIG. 3.

4) Assistive Knee Torque: Although the PT might need to support the lower knee, in data collection sessions there were very few instances where the PT was exerting force on the force sensor at the shank. Therefore, the sagittal forces applied to the thigh (Fₚₜ as shown in FIG. 3) were considered. This force was always applied in the depicted direction which results in positive torque. Using the force sensor, both the magnitude and center of pressure (CoP) of the exerted force on the thigh can be calculated. The effect of this force can be represented by a knee and ankle joint torque. These two torque components are coupled as they are the result of the same force Fₚₜ. For simplicity, the knee torque component τ= [symbol] |Fₚₜ| is considered, as the ankle joint torque component can be calculated based on the learned torque τ, given kinematic parameters including knee angle displacement θ and shank angle displacement α (FIG. 3) and shank length [symbol] .

Note that the gripping and torsional forces can affect sensor readings but are treated as noise. Due to the low relative magnitude and different frequency, which is partially removed by filtering, gripping and torsional forces are considered to have a negligible impact on the final knee torque calculation.

5) Ratings: In each session, the PT facilitating the patient's lower limb rated the quality of their assistance during each gait cycle as “good,” “ok” and “bad,” while the other PT input the rating into the system by pushing the corresponding button on a detachable remote connected to the backpack. Only gait cycles rated as “good” (more than 70% of the cycles), were included in the data set for analysis and learning.

B. Manual Gait Rehabilitation with Hemi-Paretic Patients

Patients with hemiparesis often present with a slow, asymmetrical gait pattern, which may be due to knee instability and inadequate weight-shifting. During the assessment, knee instability is often confirmed when weakness in hamstrings or quadriceps muscles and/or proprioceptive deficits are identified. A hemiparetic gait includes knee hyperextension during initial loading due to the lack of control of the quadriceps muscles. At mid-stance, overall muscle weakness can cause increased hip and knee flexion, which may be observed as knee buckling. During terminal stance, knee hyperextension may occur if the hamstrings are too weak to counteract the quadriceps to slow down knee extension and the knee may quickly hyperextend.

Patients with hemiparesis may compensate for weakness at different joints during a gait cycle to improve stability. In the stance phase, two common strategies to prevent knee hyper-extension or buckling are to limit stance time by decreasing the weight-shifting on the involved extremity or to maintain excessive knee flexion. These gait compensations often result in an asymmetrical gait pattern with decreased step length on the contralateral side and altered joint kinematics throughout the lower extremity. Over time, compensatory movement patterns may reinforce the abnormal tone and movement of the involved side. In summary, gait abnormalities related to knee instability on the paretic side can be categorized as:
Knee hyperflexion in mid-stance
Knee hyperextension in terminal stance.
Late loading and early unloading (insufficient weight-shift) from the paretic side to the non-paretic side (limiting stance time on the paretic side)

These gait impairments often result in decreased step length on the non-paretic side, asymmetric gait, and altered lower-limb joint kinematics on both sides. It should be noted that a patient may not have all three of these features.

The stance phase is key to facilitating a symmetrical gait pattern since increased stance time on the involved side allows the patient time to take a normal step with the non-involved lower extremity. Functional stance is achieved when appropriate weight is shifted onto the involved lower extremity while stabilizing the knee, which may require manual support and facilitation of the quadriceps or hamstrings. Timing is critical, and facilitation is performed as needed to achieve proper joint kinematics during each phase of stance. Repetition of proper gait mechanics is needed for motor learning to occur, which is important as learning is required for neural adaptation. On the other hand, active participation of the patients is essential to prevent over-reliance on the PT and automation.

Consequently, the main training strategies of the PTs would be to 1) facilitate the knee extension during mid-stance (specifically when reaching the maximum weight-acceptance) and 2) increase the stance time (weight-shifting period) on the paretic side to allow enough time for the non-paretic side to take a normal step. The rest of this section shows characterizations of the patient's gait patterns and gait rehabilitation features described above through visualizations of the collected data.

### C. Characterization of Patients' Gait

The MMT test results (in Table I) are used to characterize the impairment levels of the four patients. MMT scores range from 0 to 5, with 0 being no contractions (on the muscles), and 5 being full range of motion against significant resistance. Therefore, P7 has the healthiest gait among other patients. P3 has the lowest scores on knee flexion and hip extension. Based on MMT results, the patients can be grouped into lower and higher muscular weakness (LMW and HMW) groups. As shown in Table I, P7 and P5 are in LMW, and P6 and P3 are in HMW.

FIGS. 4A and 4B show a visualization of the collected data for two patients. The solid lines represent the mean gait cycle of the particular data, while the shaded region shows the ± one standard deviation. NH (dotted line) refers to the standard knee angle trajectory of healthy individuals in over-ground walking at their natural speed. For generating this result and the rest of the disclosure, 55±8, 44±18, and 35±4 (averaged among the patients) cycles are used for NT, PT1, and PT2 sessions, respectively. Bar plots show the gait phases for each session. GP stands for gait phase. Annotations indicate (I) excessive knee flexion in early MST and maximum load, (II) small or no flexion entering MST, (III) knee extension at HS, and (IV) first peak (maximum load).

In particular, FIGS. 4A and 4B show the knee angles and weight-shift of the patients, as well as the assistive torque for analyzing the PT strategies corresponding to abnormal gait patterns. This figure includes the data for P6 and P7, each representing one muscular weakness group. To provide a reference for comparison, a knee angle pattern of over-ground walking of healthy individuals in their natural speed is also provided, by averaging over 50 subjects data using a public dataset. Overall, there were no cases of severe knee buckling and hyperextensions in the NT sessions, as the patients all had MMT scores >2+ and were evaluated by the PTs to be able to ambulate with minimal assistance or less. Nevertheless, for P7 excessive knee flexion angle is observed in early mid-stance and at maximum weight-acceptance (as labeled in the knee angle plot for P7 in FIG. 4B), which can be associated with the weakness of knee extensors. On the other hand, small or no knee flexion is observed when entering mid-stance for P6 (as labeled in the knee angle plot for P6 in FIG. 4A), which is another compensation strategy to stabilize the knee with weakness of knee extensors at mid-stance. Knee extension at heel-strike as a compensatory mechanism for the weakness of planter-flexor muscles is also observed in P6.

All patients showed similar irregular GRF patterns. The first peak, which represents the total weight acceptance, happens relatively late at the mid-stance, and the second peak, which is expected to be observed at terminal-stance/pre-swing, is blended into the first one and decreased in magnitude, shown in FIGS. 4A and 4B (second row). Similar patterns were also seen in previous studies of post-stroke patients. This can be explained partly by the lack of proper heel-strike and push-off due to ankle dorsiflexion and plantarflexion weakness.

### D. Gait Rehabilitation (PT Strategy) Characterization

Here, the outcome of each gait rehabilitation session is examined, highlighting the similarities and differences between the strategies employed by the PTs. According to the assistive torque plots in FIGS. 4A and 4B (third row), generally lower magnitude torque is exerted for LMW patients, as expected. Peak torque is mostly located at 25% to 50% gait cycle, near where the maximum weight-acceptance in mid-stance takes place. This observation demonstrates an assistance strategy where the PT facilitates weight shifting onto the paretic side while stabilizing the knee to prevent buckling.

One goal of the PT is to increase stance time on the paretic side. As seen in Table II, the stance time is increased in the training sessions. This increase is less frequently observed for P7, which could be related to the low impairment level of this patient compared to the others. More specific outcomes can also be observed for other patients. For example, P7's excessive knee flexion in mid-stance is corrected in the training sessions. It should be noted that motor skill acquisition through manual gait rehabilitation is gradual and requires multiple sessions for the patient's joint kinematics to have a significant shift towards healthier gait patterns.

### IV. Virtual Impedance Learning

The work thus far has provided a foundation for understanding and analytically evaluating how the PT provides assistance to address different types of gait behaviors. A low-dimensional model of PT decision-making is presented, driven by insight gained from the previous analysis. The intended result of the algorithm is to produce a predictive model, that follows principles of gait rehabilitation, faithfully captures high-level PT strategies, and provides a compliant and safe mode of control for lower-limb exoskeletons. The remainder of this section will first provide an overview of the insights and novel aspects incorporated in this algorithm. Then, a brief formulation of the algorithm implemented by the predictive model 130 in FIGS. 1A and 1B will be given and followed by an analysis and results of its implementation.

### A. Incorporating Insights

The analysis in Sec. Ill informs the system of rehabilitation insights by providing a quantitative description of impairment that aligns with the identified deterministic features of PT assistance; patient kinematics and weight-shift. Instead of solely using knee angle as the descriptor of kinematics, this insight is expanded upon by first assuming that there are relatively small changes in ankle flexion and torso angle due to the ankle being constrained with an ace wrap and the torso being supported by a second therapist during data collection. Therefore, the shank displacement angle α and thigh displacement angle β are selected as features that describe patient kinematics, since they explicitly account for knee angle and include partial information about hip and ankle kinematics. Weight-shift w is also selected as a feature due to the aforementioned significance in facilitating increased stance time, as seen in Table II, to prevent asymmetric gait patterns. Lastly, percent progression through the stance phase t is selected as the time-indexing feature to account for PT assistance being specific to different gait sub-phases and its invariance to different walking speeds. These features are selected due to their determinism in the observed assistance and they allow the system to identify classes of gait behaviors that enable characterization of unique assistive responses from the PT.

TABLE II
Patient Stance Times

Session

| Patient ID (P) | NT | PT1 | PT2 |
|---|---|---|---|
| 3 | 1.17 ± 0.13 | 1.29 ± 0.22 | 1.50 ± 0.46 |
| 5 | 1.51 ± 0.34 | 2.33 ± 0.68 | 2.13 ± 0.78 |
| 6 | 2.08 ± 0.46 | 2.48 ± 0.57 | 2.94 ± 0.45 |
| 7 | 1.28 ± 0.16 | 1.32 ± 0.21 | 1.38 ± 0.13 |

The next insight considered is that PT assistance is applied to intentionally move the patient into preferred states, as seen in FIGS. 4A and 4B, when comparing assisted and unassisted sessions. Also, the PT is not simply correcting the patient's current state, and is instead facilitating the forward progression of the patient through the gait cycle. Given the fairly safe assumption that the PT's assistance objective was met during strides rated as “Good”, these preferred future states can be defined as the PT's goal state and as the attractor of the virtual impedance system.

One caveat of this definition for the attractor is that it prevents the present model of PT behavior from describing torque components that are not directly determined by the displacement between the patient's current and future states. To address this, the PT may use strategies that generate forces that do not directly contribute to the patient's progress through the previously defined states. One such strategy can be described as proactively bracing the knee to prevent knee instability. These types of strategies are included in the algorithm by reproducing the unexplained assistance, described by the residual torque from the model of interaction, found during the optimization of the impedance gains.

### B. Mathematical Formulation

In this disclosure, referring to FIGS. 1A-3, PT's assistive torques to the knee will be described by the following virtual impedance model formulation:

$${τ}_{t} = ( {y}_{t} - {x}_{t} ) - {K}_{t}^{𝒱} {\dot{x}}_{t} , \qquad ( 1 )$$

where [symbol] and

$${K}_{t}^{𝒱}$$

are the continuous time-varying stiffness and damping values, τₜ is the torque applied by the PT around the patient's knee, xₜ=[wₜ, αₜ, βₜ]^{T} is the state of the virtual impedance system, and yₜ is the future-state attractor at a percent progression through the stance phase t. During reproduction, an auxiliary term rₜ is added to Eq. (1) as an estimate of the residual torque, which is discussed in Sec. IV-A.

The future-state attractor yₜ is simply the state xₜ that will be observed in an additional t_{f} percent stance phase in the future where yₜ=[wₜ₊ₜ_{_{f}}, αₜ₊ₜ_{_{f}}, βₜ₊ₜ_{_{f}}]^{T}. Here, t_{f} is a design parameter manually tuned for each PT-P pair to optimize the performance objective of f(t_{f})=0.5(r²)+0.5(RMSPE) where r² and RMSPE are the coefficient of determination and root mean squared percent error, respectively, of the final torque prediction generated by this algorithm. Intuitively, the subscript t+t_{f} describes the state that is t_{f} percent gait phase in the future relative to the state at time t. The mean value for the time-offset was found to be t̅_{f}=28.25% with a standard deviation of σₜ_{_{f}}=10.9%. Intuitively, this suggests that the attractive state of the virtual impedance system was found to be, on average, the patient state in the next sub-phase of the gait cycle.

An estimate of the observed assistance can be recovered once the time-varying prediction of residual torque rₜ, attractor yₜ, stiffness [symbol] and damping

$${K}_{t}^{𝒱}$$

gains are known. Therefore, the objectives of the learning step are to 1) create a gait pattern classifier such that a learned assistance strategy is uniquely defined in response to each class, 2) parameterize stiffness gains [symbol] , damping gains

$${K}_{i}^{𝒱} ,$$

and residual torques

$${K}_{i}^{r}$$

for each i-th classification of gait patterns, and 3) learn a predictive model for yₜ.
1) Training: Given a set of demonstrations d=[t, xₜ, yₜ, τₜ], two additional samples of previous states (xₜ₋ₜ_{ₚ}

$$for i ∈ [ 1 , k ] :$$

$${min}_{, {K}_{i}^{𝒱}} {‖ {p}_{t , i} ( {r}_{t , i} ) ‖}_{2}$$

$$s . t . {r}_{t , i} = ( {μ}_{i}^{y} - {x}_{t} ) - {K}_{i}^{𝒱} ( {\dot{x}}_{t} ) - {τ}_{t} ,$$

$${K}_{lb}^{P} \geq {K}_{i}^{P} \geq {K}_{ub}^{P} ,$$

$${K}_{i}^{V} \geq 0 ,$$

where [symbol] and

$${K}_{i}^{𝒱}$$

are the stiffness and damping gains found for each i-th Gaussian component,

$${μ}_{i}^{y}$$

is attractor's dimension along the GMM's center, p_{t,i} is the posterior probability from the GMM encoding, and

$${K}_{lb}^{P}$$

band

$${K}_{ub}^{P}$$

are the lower and upper bounds of feasible stiffness values. During optimization, the mean residual torque is found for each cluster

$${K}_{i}^{r}$$

by taking the weighted average of r_{t,i} for every time-step:

$${K}_{i}^{r} = {\sum }_{t = 0}^{{n}_{t}} {p}_{t , i} ( {r}_{t , i} )$$

where nₜ is the number of time steps.

2) Reproduction: Reproduction begins with an encoded GMM in the form

$$( {μ}_{i} , {Σ}_{i} ) and , {K}_{i}^{𝒱} , and {K}_{i}^{r}$$

vectors for i∈{1, 2, . . . , k}. Gaussian mixture regression (GMR) is performed with the observed predictors [symbol] =[t, xₜ, xₜ₋ₜ_{ₚ}, xₜ₋₂ₜ_{ₚ}]=to find the probability ĥ_{t,i} that a sample from [symbol] belongs to each Gaussian component. Then,

$${μ}_{i}^{y} , , {K}_{i}^{𝒱} and {K}_{i}^{r}$$

are mixed according to ĥ_{t,i} by taking the weighted sum of each component as follows:

$${[ {\hat{y}}_{t} , , {K}_{t}^{𝒱} , {r}_{t} ]}^{T} = {\sum }_{i = 1}^{k} {{\hat{h}}_{t , i} ( ) [ {μ}_{i}^{y} , , {K}_{i}^{𝒱} , {K}_{i}^{r} ]}^{T} , \qquad ( 2 )$$

where ŷₜ is the predicted attractor and rₜ is the estimate of the unexplained assistance for each continuous time-step t. The final estimate of the observed torque \hat{τ}ₜ is recovered by substituting these parameters into Eq. (1) and then adding rₜ:

$${\hat{τ}}_{t} = ( {\hat{y}}_{t} - {x}_{t} ) - {K}_{t}^{𝒱} {\dot{x}}_{t} + {r}_{t} . \qquad ( 3 )$$

### C. Analysis

This analysis will first evaluate the trained model on its ability to appropriately classify patient gait patterns and characterize PT impedance strategies. A hard clustering procedure, where samples are classified according to the Gaussian component with the highest posterior probability, will be performed on the testing data set. Qualitative analysis will then be used to determine if the GMM can adequately identify unique presentation of gait patterns. Stiffness gains are then compared between classes to see if the characterized impedance strategies align with what can be expected from a PT responding to different classes. For example, more aggressive (higher) stiffness gains can be expected during classes that characterize significant compensatory behaviors like knee hyperextension during mid-stance. More aggressive stiffness gains would then align with increased PT intervention to facilitate desired assistance outcomes. Here, the unsigned magnitude of the impedance gains is examined due to the ambiguity introduced by allowing [symbol] to be positive or negative such that [symbol] (yₜ−xₜ) generates a positive torque. In place of analyzing all patient gait pattern classifications, which would be exceedingly lengthy, a more in-depth analysis of P6's session with PT2 will be provided as an example.

The algorithm's ability to characterize the observed assistance using the interaction model in Eq. (1) is evaluated by analyzing the residual torque estimated during optimization

$${K}_{i}^{r}$$

and the residual torque included in the reproduction rₜ. This will help identify the degree of reliance on the residual torque estimation during reproduction. To provide additional support for the validity of the trained model, the GMR's ability to predict yₜ will also be evaluated using the coefficient of determination r² and root mean squared error RMSE. Similarly, r² and RMSE are reported as a baseline for the predictive power of \hat{τ}ₜ.

The overall performance of the algorithm is evaluated by how well the reproduced assistance matches key characteristics of PT behavior. Performance in replicating the magnitude of observed assistance is evaluated using the peak torque τₚ and impulse/for every j-th stride. J can be found by integrating τₜ over the stance duration for each stride as

$$J = {\int }_{{t}_{s}^{0}}^{{t}_{s}^{1}} {τ}_{t} ( {t}_{s} ) {dt}_{s}$$

where

$${t}_{s}^{0} and {t}_{s}^{1}$$

are the time at 0% and 100% of each stride's stance phase.

The timing of peak assistive torque tₚ, in percent stance phase, is evaluated due to the relevance of reproduction accuracy during the most safety-critical time; mid-stance. Additionally, an approximation of cumulative timing accuracy is given using a dynamic time warping (DTW) algorithm. This algorithm minimizes the Euclidean distance between the normalized signals by stretching t. The average delay over all strides [symbol] ₜ is then calculated and rescaled for every time-index t∈[0, 100], which gives an approximate delay of the reproduced assistance. For brevity, only the mean delay [symbol] is reported instead of the delay at every time step.

The PT may apply pseudo-random perturbations to gauge the current status of the unassisted gait, prevent overreliance on assistance, or automation of the task by the patient, which have all been shown to negatively impact rehabilitation outcomes. Therefore, the standard deviation of both peak torque σ_{τ}_{ₚ} and impulse σⱼ is analyzed to evaluate the ability to emulate how much the PT adapts or changes their behavior.

Lastly, the performance of the algorithm is viewed in a more general sense by looking at the high-level behaviors of the PT. This is done to investigate an emergent behavior of the algorithm that shows a loss of information describing stride-to-stride variance. This is partly to be expected due to the abstraction of trajectories with high variance into a finite set of components during GMM encoding. Therefore, increased performance when evaluating the general response of the PT is expected. Here, “general response” refers to the mean torque over all strides for each t within a PT-P session. To investigate this expectation, the same performance criteria mentioned above are computed for the general response of the PT.

For valid comparison between MW groups, which require different assistance behavior, metrics may be reported as the mean percent error of the reproduced assistance defined as RMSPE when necessary.

### D. Results

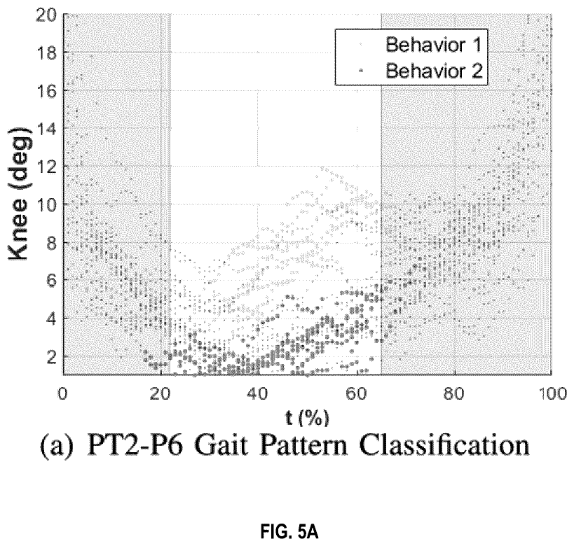

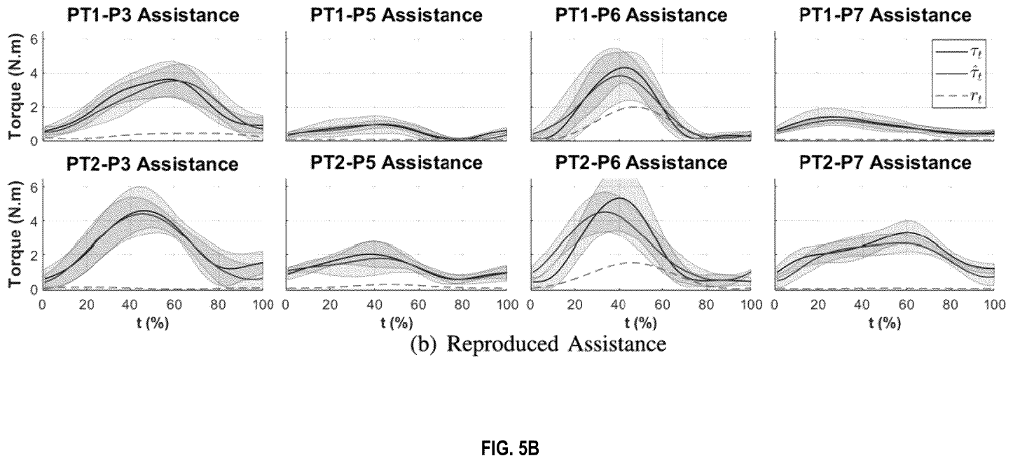

The algorithm was observed to accurately emulate high-level PT assistance behaviors while still capturing some stride-to-stride variation. For each PT-P session, the mean torque and standard deviation for observed and reproduced assistance can be found in FIG. 5B. The remainder of this section will use these results, in addition to the remaining analysis strategies in the previous section, to highlight relevant observations and evaluate the system from multiple perspectives.

1) Gait Pattern Classification: The algorithm successfully demonstrated the ability to classify different types of patient gait behaviors and characterize appropriate impedance strategies in response. FIG. 5A shows the results of a hard clustering procedure on samples (N=2100) from PT2-P6's training session. Here, the GMM can be seen differentiating between P6's knee flexion by classifying separate samples when the patient presented with moderate knee flexion (Behavior 1) and full knee extension (Behavior 2) during mid-stance. Consequently, unique impedance strategies were able to be characterized in response to Behavior 1 (described by a single cluster) with | [symbol] |=[0.03, 0.38, 4.32] and Behavior 2 (described by two clusters) with an average | [symbol] |=[1.61, 1.16, 5.22] for their respective thigh, shank, and weight-shift stiffness gains. Significant (p=3.3e-10) difference between the mean assistance in Behavior 1 (N=272, M=3.0, SD=1.1) and Behavior 2 (N=313, M=3.7, SD=1.5) was observed. This suggests that PT2 provided more assistance during Behavior 2 which can be interpreted as a response to when the patient demonstrates less “appropriate knee-flexion” (i.e., hyperextended) than Behavior 1. Consequently, this aligns with the higher stiffness gains found for Behavior 2 indicating a more aggressive torque interaction when pursuing the intended future state of the patient.

2) Residual Estimation: The model was able to explain the majority of the observed torque with the modified interaction model as in Eq. 3 during optimization. A mean residual of r̅ᵢ=0.08N·m was found for all sessions with the exception of τ̅ₜ, an outlier, that had r̅ᵢ=0.53N·m. This is still believed to be sufficiently accurate with 11% of the observed peak torque being unexplained. FIG. 5A shows the residual torque component rₜ (dashed, red line) included in the final reproduction of τ̅ₜ. Observations of rₜ in FIG. 5A support the assumption that it is reproducing unmodeled assistance strategies like knee bracing. When present, rₜ occurs most significantly during periods of maximum weight acceptance which requires a higher magnitude of assistance from the PT. The timing of increased rₜ aligns with what can be expected from these strategies since the higher requirement for assistance also applies to these unmodeled strategies. Further evidence supporting that rₜ represents these strategies is given by the absence of significant rₜ in some patients, particularly those in the LMW group. This suggests that rₜ is caused by a systemic perturbation from PT-P interaction and not by the sensing system, otherwise, this increased rₜ is likely to be observed throughout all sessions.

3) Attractor Prediction: Accurate prediction of the patient's future state was observed by the trained model for all sessions. The GMR's prediction of the attractor obtained an average of r²=[0.79, 0.73, 0.84] and RMSE=[2.65, 2.75, 0.13] for thigh, shank, and weight-shift, respectively.

4) Torque Prediction: The predicted assistance had an average r²=0.53 and RMSE=0.81, 0.41 for HMW and LWM groups, respectively. Notable differences in the performance in r² were observed between MW groups (r²=0.61 and r²=0.45 for HMW and LMW, respectively), while little difference was observed for RMSPE for any group.

5) Magnitude of Assistance: The magnitude of the reproduced assistance achieved a RMSPE of 28.00% and 24.91% for tₚ, and J, respectively.

6) Variation in Assistance: For all sessions, reproduction showed lower variation than the observed response in tₚ and J with a mean percent error of approximately 15% for both. This is with the exception of the variation in tₚ for the PT1-P6 and PT2-P5 sessions where the reproduced assistance showed 20% and 8% more variation than the observed assistance, respectively. Between PTs, the variation for tₚ was more accurate for PT2's when compared to PT1's with 8% and 22% error, respectively. Less significant variance in tₚ was observed between MW groups with 12% and 18% error for the LMW and HMW group, respectively.

7) Timing: The algorithm displayed accurate timing of assistance with a mean error of D=2.49% and tₚ=7.28% over all sessions. More accurate prediction of peak timing and delay was observed for the HMW group when compared to the LMW group. No differences in timing were observed between PTs.

8) High-Level Behaviors: The analysis thus far indicates the moderate ability for the algorithm to emulate PT behavior on a stride-to-stride basis. However, analysis of the general response of the PT, illustrated by the solid lines in FIG. 5B, shows an increased performance of the algorithm when evaluated in this scope. An improvement, on average across all sessions, was seen in mean regression metrics (r²=0.92 and RMSPE=0.32%), mean timing (D=2.6% and tₚ=3.25% error) and in mean peak torque tₚ=10.1%. These results indicate that the algorithm is primarily able to capture high-level PT behavior while being able to explain some of the variations between strides. Further explanation of the causes and implications of this is given in the next section.

### V. Discussion

In this disclosure, significant effort was invested into creating an insight-driven and data-informed foundation for learning PT assistance behaviors. The analysis in Sec. IV showed that a unique and diverse set of gait behaviors and assistive responses emerged from patients that would generally be considered to have the same level of impairment. Although the presentation of impairment was unique for each patient, most observations aligned with insights from previous post-stroke rehabilitation studies, such as excessive knee flexion during mid-stance or episodes of knee hyperextension. This observation shows the importance of learning assistance strategies from PT-P interactions.

The system incorporates the feature of weight-shift to emulate qualities of the PT. While weight-shift is generally an important and well-studied phenomenon in gait rehabilitation of post-stroke patients, it has received far less attention when developing wearable robots. Weight-shift in conjunction with a future-state attractor and more common kinematic features (thigh and shank), to generate positive results which match the magnitude and timing of PT assistance. Timing of the assistance is especially critical for effective rehabilitation and reduction of metabolic cost. Success in these areas implies that a lower-limb exoskeleton would be able to provide the appropriate assistance to the patient.

However, the algorithm showed some challenges in reproducing the nuances in stride-to-stride assistance variation. This observation is likely due to a combination of three factors: 1) the PT may be unable to assist in a perfectly consistent way due to stochasticity of human-human interaction and the associated challenges with manual gait rehabilitation, 2) inherent limitations in the sensing system may cause variation in observed data, and 3) the abstraction of high variance torques to a cumulative model of behavior during GMM encoding causes loss of information describing variation between strides in an attempt to be robust against over-fitting and noise. Alternatively, the model may be missing the deterministic feature(s) that explain stride-to-stride variations such as adaptive PT assistance as the patient fatigues or improves. However, it is important to be cautious when adding additional features to balance the trade-off between 1) a low-dimensional and generalizable model, 2) adding cumbersome sensors that interfere with patient rehabilitation, and 3) possible introduction of noisy inputs.

### A. Implications

The system functions as a high-level decision framework that generates a continuous control input (reproduced torque) in a way that emulates assistive PT behaviors. Any arbitrary low-level controller (i.e., PID) can then use this control input to actuate a 1-DoF lower-limb knee exoskeleton to provide assistive knee torques. Specifically, this work shows how additional expert PT knowledge and clinical insights can be identified and integrated into a high-level decision model. Done successfully, this implies that a more robust, effective, and personalized robotic assistance can be generated. Furthermore, the characterization of discrete impedance strategies in response to classes of gait behaviors reduces the learning problem from encoding the assistive response for every possible continuous-state combination to one that is tractable, computationally efficient, and more robust against over-fitting. Opportunity for generalizing to new patients is also present given the fairly safe assumption that assistance strategies do not significantly change between patients with similar gait behaviors. Therefore, once a sufficient amount of data is collected to characterize common gait patterns, the trained model will require little to no additional training when being used to assist a new patient.

A common limitation in studies like this is the limited access to patients, especially amid the COVID-19 pandemic. With a sample size of four patients over eight sessions, conclusions about statistical significance are difficult to make for both the biomechanical characterization and algorithm performance, and more trials are needed to be conducted in the future.

One significant limitation of the wearable sensor system is its ability to capture the information available to PT. A vast set of observations, such as verbal communication, context, and full-body patient kinematics are all exceedingly difficult to capture in full. Although the latter can be addressed with additional sensors, a trade-off between the resolution of data and interference in rehabilitation sessions must be considered such that it does not decrease the validity of the data or detract from the patient's rehabilitation outcomes. However, one addition that can be developed is capture of joint kinematics on the non-affected side to observe the symmetry of the patient's gait, which is an important gait rehabilitation outcome. Similarly, estimating the center of mass trajectory could be advantageous for a more detailed description of how weight is shifted.

As for the algorithm, the primary goal of future work is to implement a more supervised approach to the GMM encoding. Although the GMM was successful in classifying gait behaviors, placement of cluster centers could more directly align with how a PT would interpret these classes. This would result in clearer boundaries between class definitions and enable a more accurate representation of the PT decision-making model. Finally, dynamically adjusting the time offset t_{f} of the future-state attractor would likely help the model better align with true modes of PT decision-making.

For successful implementation of the presented model for robot-aided training, the validity of the model in reproducing PT behavior should be further tested with a large number of patients and PTs. Additionally, the reliability and safety of a control model implemented using the system should be verified when presented with perturbations that resemble unseen but possible events during the gait rehabilitation, such as tripping.

This disclosure presented a predictive model for lower-limb exoskeletons that adopts an insight-driven and data-informed approach to modeling PT assistance. Data were collected during live gait rehabilitation sessions at a resolution not commonly found in PT behavioral modeling. Analysis of this data allowed for a nuanced description of patient impairment in terms of practical rehabilitation principles found in the surrounding literature. A novel LfD impedance algorithm was then implemented and observed to successfully characterize patient gait patterns and capture high-level PT response strategies. Implications of this research provide an opportunity for an insightful model-based approach to controlling rehabilitation robotics. Plans for implementing this modeling framework in a lower-limb exoskeleton is in place to evaluate if the system translates to a real rehabilitation setting.

The functions performed in the processes and methods may be implemented in differing order. Furthermore, the outlined steps and operations are provided as examples, and some of the steps and operations may be optional, combined into fewer steps and operations, or expanded into additional steps and operations without detracting from the essence of the disclosed embodiments.

### VI. Computer-Implemented System

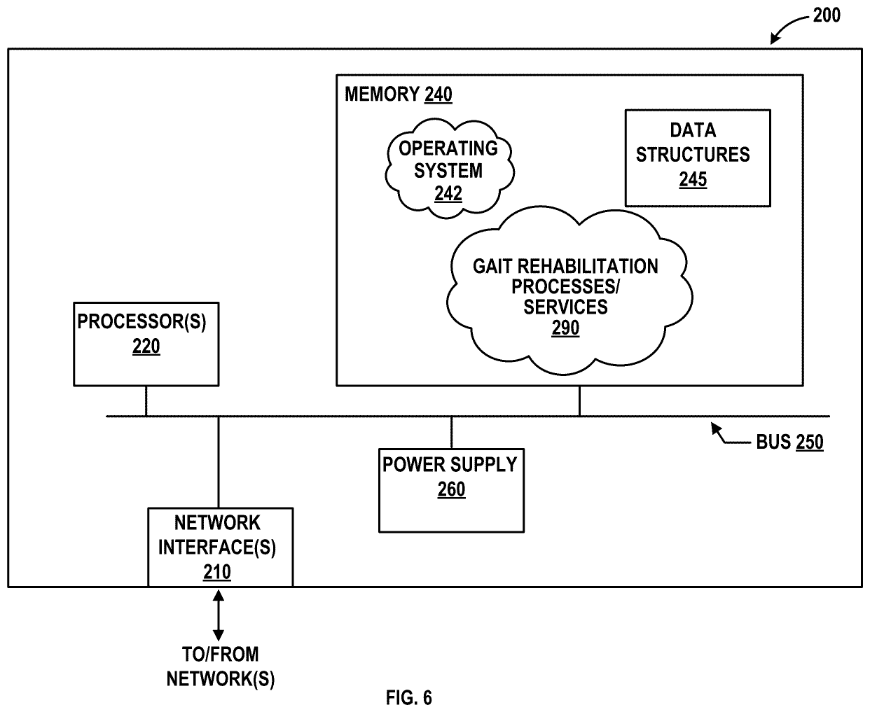

FIG. 6 is a schematic block diagram of an example device 200 that may be used with one or more embodiments described herein, e.g., as a component of system 100 shown in FIG. 1A implementing aspects of the framework shown in FIG. 1B.

Device 200 comprises one or more network interfaces 210 (e.g., wired, wireless, PLC, etc.), at least one processor 220, and a memory 240 interconnected by a system bus 250, as well as a power supply 260 (e.g., battery, plug-in, etc.).

Network interface(s) 210 include the mechanical, electrical, and signaling circuitry for communicating data over the communication links coupled to a communication network. Network interfaces 210 are configured to transmit and/or receive data using a variety of different communication protocols. As illustrated, the box representing network interfaces 210 is shown for simplicity, and it is appreciated that such interfaces may represent different types of network connections such as wireless and wired (physical) connections. Network interfaces 210 are shown separately from power supply 260, however it is appreciated that the interfaces that support PLC protocols may communicate through power supply 260 and/or may be an integral component coupled to power supply 260.

Memory 240 includes a plurality of storage locations that are addressable by processor 220 and network interfaces 210 for storing software programs and data structures associated with the embodiments described herein. In some embodiments, device 200 may have limited memory or no memory (e.g., no memory for storage other than for programs/processes operating on the device and associated caches). Memory 240 can include instructions executable by the processor 220 that, when executed by the processor 220, cause the processor 220 to implement aspects of the system 100 and the methods outlined herein.

Processor 220 comprises hardware elements or logic adapted to execute the software programs (e.g., instructions) and manipulate data structures 245. An operating system 242, portions of which are typically resident in memory 240 and executed by the processor, functionally organizes device 200 by, inter alia, invoking operations in support of software processes and/or services executing on the device. These software processes and/or services may include gait rehabilitation processes/services 290, which can include aspects of methods and/or implementations of various modules described herein. Note that while gait rehabilitation processes/services 290 is illustrated in centralized memory 240, alternative embodiments provide for the process to be operated within the network interfaces 210, such as a component of a MAC layer, and/or as part of a distributed computing network environment.

It will be apparent to those skilled in the art that other processor and memory types, including various non-transitory computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the gait rehabilitation processes/services 290 is shown as a standalone process, those skilled in the art will appreciate that this process may be executed as a routine or module within other processes.

### VII. Method

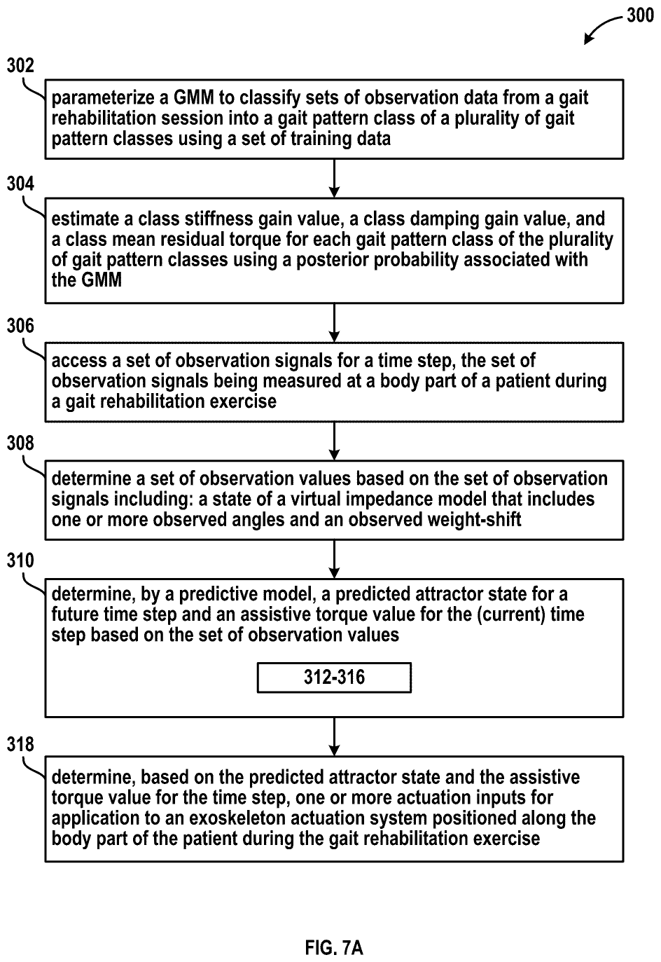

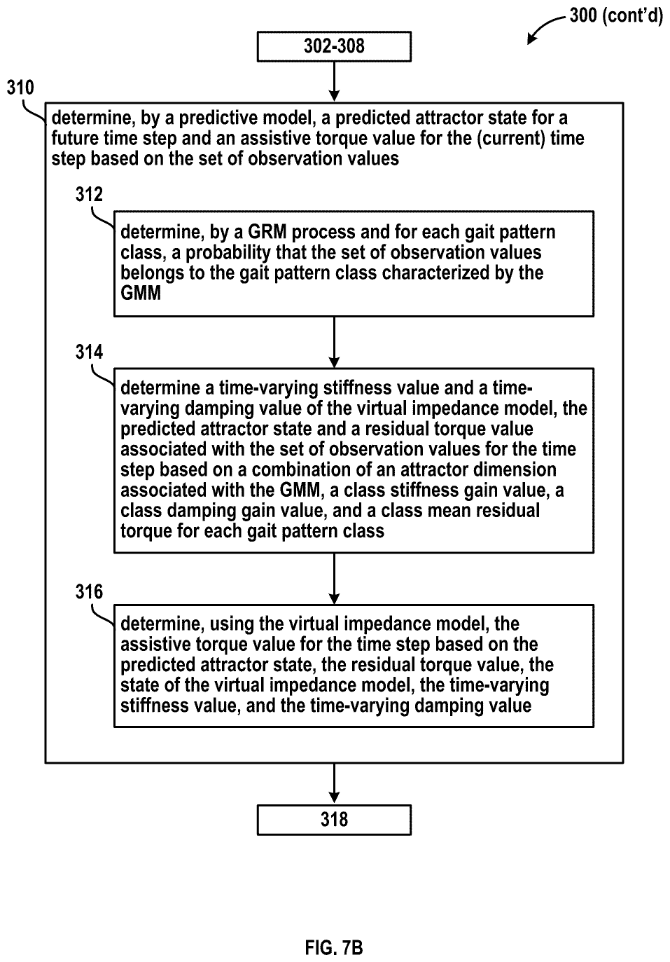

FIGS. 7A and 7B show a method 300 associated with the system 100 outlined herein with respect to FIGS. 1A and 1B and Sections I-III which may be implemented using the computing device 200 of FIG. 6. Method 300 can correspond with gait rehabilitation processes/services 290 of FIG. 6.

Steps 302 and 304 are training steps associated with characterization of a Gaussian Mixture Model that aims to identify appropriate impedance learning parameters for reproducing observed PT-applied torque behaviors, which are dependent upon a portion of a “walk cycle” or “gait pattern class” associated with current and expected future observation data.

Step 302 includes parameterizing a Gaussian Mixture Model to classify sets of observation data from a gait rehabilitation session into a gait pattern class of a plurality of gait pattern classes using a set of training data (“Feature Extraction”, “Encode GMM” blocks in FIG. 1B). Step 304 includes estimating a class stiffness gain value [symbol] , a class damping gain value

$${K}_{i}^{v} ,$$

and a class mean residual torque

$${K}_{i}^{r}$$

for each gait pattern class of the plurality of gait pattern classes using a posterior probability p_{t,i} associated with the Gaussian Mixture Model (“Impedance Optimization block in FIG. 1B).

The observation data can be measured during the gait rehabilitation exercise by a plurality of sensors including: one or more pressure sensor arrays associated with the body part and being operable for measuring components of an assistive force value (e.g., a sagittal force Fₚₜ applied to the body part by a physical therapist) associated with the body part and a ground reaction force (e.g., net ground reaction force F_{g} applied to foot by the ground) associated with the body part during the gait rehabilitation exercise. The ground reaction force F_{g}, along with a known weight of the patient F_{w}, can be used to obtain a weight shift value w.

The plurality of sensors can include an inertial measurement sensor associated with the body part and being operable for measuring one or more kinematic values (of the observation data) associated with the body part, including a shank displacement angle α and a thigh displacement angle β. The shank displacement angle α and the thigh displacement angle β can be used to obtain a knee angle θ.

Steps 306-318 are steps associated with real-time torque behavior prediction which can be applied following development and parameterization of the Gaussian Mixture Model in step 302 and optimization of impedance parameters in step 304.

Step 306 includes accessing, by a processor in communication with a memory, a set of observation signals for a time step t, the set of observation signals being measured at a body part of a patient during a gait rehabilitation exercise.

Step 308 can include determining a set of observation values [symbol] [symbol] =[, xₜ, xₜ₋ₜ_{ₚ}, xₜ₋₂ₜ_{ₚ}] (where xₜ=[wₜ, αₜ, βₜ]^{T}) based on the set of observation signals, the set of observation values [symbol] including: a state xₜ of a virtual impedance model that includes one or more observed angles (shank displacement angle α, thigh displacement angle β) and an observed weight-shift w associated with the body part of the patient for the time step; the virtual impedance model relating the state xₜ to a time-varying stiffness value [symbol] , a time-varying damping value

$${K}_{t}^{v} ,$$

the assistive torque value \hat{τ}ₜ and the predicted attractor state ŷₜ.

The set of observation signals can be measured during the gait rehabilitation exercise and can include one or more kinematic values associated with the body part, including the shank displacement angle α and the thigh displacement angle β which can be used to obtain a knee angle θ. Further, the set of observation signals [symbol] can include the weight shift value w which can be obtained using the ground reaction force F_{g}, along with a known weight of the patient F_{w}.

The shank displacement angle α, the thigh displacement angle β, and the weight shift value w for a given time step t can be collected into the state xₜ. Additionally, states xₜ₋ₜ_{ₚ} and xₜ₋₂ₜ_{ₚ} collect these values for past time steps (e.g., xₜ₋ₜ_{ₚ} and xₜ₋₂ₜ_{ₚ}).

Step 310 includes determining, by a predictive model implemented at the processor, a predicted attractor state ŷₜ˜yₜ=[wₜ₊ₜ_{_{f}}, αₜ₊ₜ_{_{f}}, βₜ₊ₜ_{_{f}}]^{T} for a future time step t+t_{f} and an assistive torque value \hat{τ}ₜ for the (current) time step based on the set of observation values [symbol] .

The predictive model can include the Gaussian Mixture Model which can be used with a Gaussian Mixture Regression (GMR) algorithm to identify, for each respective gait pattern class i∈{1, 2, . . . , k}, a probability ĥ_{t,i} that the set of observation values [symbol] belongs to the gait pattern class i∈{1, 2, . . . , k}.

The predictive model can also include a virtual impedance model (Eqs. (1), (3)) that correlates a current state xₜ associated with the observation signals, the predicted attractor state ŷₜ, the time-varying stiffness value [symbol] , and the time-varying damping value

$${K}_{t}^{v}$$

with the assistive torque value \hat{τ}ₜ. Further, as discussed herein with respect to Eq. (3), the virtual impedance model can further incorporate a residual torque value rₜ which can represent additional or otherwise unexplained forces that a PT may apply, such as preemptively applying a stabilizing force in case of knee instability.

Step 310 can include various sub-steps 312-316 elaborated on in FIG. 7B for determining the predicted attractor state ŷₜ˜yₜ=[wₜ₊ₜ_{_{f}}, αₜ₊ₜ_{_{f}}, βₜ₊ₜ_{_{f}}]^{T} for a future time step t+t_{f} and the assistive torque value \hat{τ}ₜ for the (current) time step based on the set of observation signals.

Step 312 can include determining, at the processor and by a Gaussian Mixture Regression process for each gait pattern class i∈{1, 2, . . . , k}, a probability ĥ_{t,i} that the set of observation values [symbol] associated with the body part of the patient for the time step t belongs to the gait pattern class i∈{1, 2, . . . , k} characterized by the Gaussian Mixture Model.

Step 314 can include determining, at the processor, a time-varying stiffness value [symbol] and a time-varying damping

$${K}_{t}^{v}$$

of the virtual impedance model, the predicted attractor state ŷₜ and a residual torque value rₜ associated with the set of observation values [symbol] for the time step based on a combination of an attractor dimension

$${μ}_{i}^{y}$$

associated with the Gaussian Mixture Model, a class stiffness gain value [symbol] , a class damping gain, value

$${K}_{i}^{v} ,$$

and a class mean residual torque

$${K}_{i}^{r}$$

for each gait pattern class i of the plurality of gait pattern classes {1, 2, . . . , k} (e.g., as in Eq. (2)).

Step 316 can include determining, at the processor and using the virtual impedance model, the assistive torque value \hat{τ}ₜ for the time step t based on the predicted attractor state ŷₜ, the residual torque value rₜ, the state xₜ of the virtual impedance model, the time-varying stiffness value [symbol] , and the time-varying damping value

$${K}_{t}^{v} .$$

Step 318 can follow step 310 (including steps 312-316 which can be sub-steps of step 310), and can include determining, at the processor and based on the predicted attractor state ŷₜ and the assistive torque value \hat{τ}ₜ for the time step, one or more actuation inputs for application to an exoskeleton actuation system positioned along the body part of the patient during the gait rehabilitation exercise. Additional steps can include applying the one or more actuation inputs to an actuator of the exoskeleton according to the assistive torque value \hat{τ}ₜ.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method, comprising:
accessing, by a processor in communication with a memory, a set of observation signals for a time step, the set of observation signals being measured at a body part of a patient during a gait rehabilitation exercise; and
determining, for a time step and by a prediction model implemented at the processor, a predicted attractor state and an assistive torque value for the time step based on the set of observation signals.

2. The method of claim 1, further comprising:
determining, at the processor and based on the predicted attractor state and the assistive torque value for the time step, one or more actuation inputs for application to an exoskeleton actuation system positioned along the body part of the patient during a gait rehabilitation exercise.

3. The method of claim 1, further comprising:
determining, at the processor and by a Gaussian Mixture Regression process, a probability that a set of observation values associated with the body part of the patient for the time step belongs to a gait pattern class characterized by a Gaussian Mixture Model, the gait pattern class being one of a plurality of gait pattern classes identified during parameterization of the Gaussian Mixture Model.

4. The method of claim 3, further comprising:
determining the set of observation values based on the set of observation signals, the set of observation values including:
a state of a virtual impedance model that includes one or more observed angles and an observed weight-shift associated with the body part of the patient for the time step; and
a time-varying stiffness value and a time-varying damping value of the virtual impedance model;
the virtual impedance model relating the state and the time-varying stiffness value and the time-varying damping value to the assistive torque value and the predicted attractor state.

5. The method of claim 4, further comprising:
determining, at the processor and based on the probability, the predicted attractor state and a residual torque value associated with the set of observation values for the time step; and determining, at the processor, the assistive torque value for the time step based on the predicted attractor state, the residual torque value, the state of the virtual impedance model, the time-varying stiffness value and the time-varying damping value.

6. The method of claim 4, the set of observation values further including:
a state of a virtual impedance model that includes one or more observed angles and an observed weight-shift associated with the body part of the patient for a first previous time step; and
a state of a virtual impedance model that includes one or more observed angles and an observed weight-shift associated with the body part of the patient for a second previous time step;
where the time step and the first previous time step are spaced equally apart from one another and where the first previous time step and the second previous time step are spaced equally apart from one another.

7. The method of claim 3, further comprising:
parameterizing the Gaussian Mixture Model to classify sets of observation data into a gait pattern class of the plurality of gait pattern classes using a set of training data, each instance of the set of training data including:
a state of a virtual impedance model for a first time step;
a state of the virtual impedance model for a second time step;
a state of the virtual impedance model for a third time step; and
an attractor state of the virtual impedance model associated with the third time step;
where the first time step and the second time step are spaced equally apart from one another and where the second step and the third time step are spaced equally apart from one another.

8. The method of claim 3, further comprising:
estimating, at the processor and using a posterior probability associated with the Gaussian Mixture Model, a class stiffness gain value, a class damping gain value, and a class mean residual torque for each gait pattern class of the plurality of gait pattern classes.

9. The method of claim 1, the set of observation signals being measured by a plurality of sensors during the gait rehabilitation exercise, the plurality of sensors including:
one or more pressure sensor arrays associated with the body part and being operable for measuring components of an assistive force value and a ground reaction force associated with the body part during the gait rehabilitation exercise; and
an inertial measurement sensor associated with the body part and being operable for measuring one or more kinematic values associated with the body part including a shank displacement angle and a thigh displacement angle.

10. A system, comprising:
a wearable sensor system having a plurality of sensors that obtain a set of observation signals during a gait rehabilitation exercise, the set of observation signals including:
a ground reaction force associated with a body part of a patient; and
one or more kinematic values associated with the body part including a shank displacement angle and a thigh displacement angle; and
a computing device in communication with the wearable sensor system, the computing device including a processor in communication with a memory, the memory including instructions executable by the processor to determine, for a time step and by a prediction model implemented at the processor, a predicted attractor state and an assistive torque value for the time step based on the set of observation signals.

11. The system of claim 10, further comprising:
an exoskeleton actuation system positioned along the body part and in communication with the computing device;
the memory of the computing device further including instructions executable by the processor to:
determine, at the processor and based on the predicted attractor state and the assistive torque value for the time step, one or more actuation inputs for application to the exoskeleton actuation system positioned along the body part of the patient during a gait rehabilitation exercise.

12. The system of claim 10, the memory further including instructions executable by the processor to:
determine, at the processor and by a Gaussian Mixture Regression process, a probability that a set of observation values associated with the body part of the patient for the time step belongs to a gait pattern class characterized by a Gaussian Mixture Model, the gait pattern class being one of a plurality of gait pattern classes identified during parameterization of the Gaussian Mixture Model.

13. The system of claim 12, the memory further including instructions executable by the processor to:
determine the set of observation values based on the set of observation signals, the set of observation values including:
a state of a virtual impedance model that includes one or more observed angles and an observed weight-shift associated with the body part of the patient for the time step; and
a time-varying stiffness value and a time-varying damping value of the virtual impedance model;
the virtual impedance model relating the state and the time-varying stiffness value and the time-varying damping value to the assistive torque value and the predicted attractor state.

14. The system of claim 13, the memory further including instructions executable by the processor to:
determining, at the processor and based on the probability, the predicted attractor state and a residual torque value associated with the set of observation values for the time step; and determining, at the processor, the assistive torque value for the time step based on the predicted attractor state, the residual torque value, the state of the virtual impedance model, the time-varying stiffness value and the time-varying damping value.

15. The system of claim 13, the memory further including instructions executable by the processor to:
a state of a virtual impedance model that includes one or more observed angles and an observed weight-shift associated with the body part of the patient for a first previous time step; and
a state of a virtual impedance model that includes one or more observed angles and an observed weight-shift associated with the body part of the patient for a second previous time step;
where the time step and the first previous time step are spaced equally apart from one another and where the first previous time step and the second previous time step are spaced equally apart from one another.

16. The system of claim 12, the memory further including instructions executable by the processor to:
parameterizing the Gaussian Mixture Model to classify sets of observation data into a gait pattern class of the plurality of gait pattern classes using a set of training data, each instance of the set of training data including:
a state of a virtual impedance model for a first time step;
a state of the virtual impedance model for a second time step;
a state of the virtual impedance model for a third time step; and
an attractor state of the virtual impedance model associated with the third time step;
where the first time step and the second time step are spaced equally apart from one another and where the second step and the third time step are spaced equally apart from one another.

17. The system of claim 12, the memory further including instructions executable by the processor to:
estimating, at the processor and using a posterior probability associated with the Gaussian Mixture Model, a class stiffness gain value, a class damping gain value, and a class mean residual torque for each gait pattern class of the plurality of gait pattern classes.

18. The system of claim 10, the plurality of sensors including:
a first inertial measurement unit positioned along an upper brace of the wearable sensor system, the upper brace being positionable along a thigh of the patient, the first inertial measurement unit being operable for measuring one or more kinematic values associated with the body part including a thigh displacement angle;
a second inertial measurement unit positioned along a lower brace of the wearable sensor system, the upper brace being positionable along a shank of the patient, the second inertial measurement unit being operable for measuring one or more kinematic values associated with the body part including a shank displacement angle; and
a pressure sensor array positioned along a shoe of the wearable sensor system and being operable for measuring a ground reaction force associated with the body part, the shoe being positionable along a foot of the patient.

19. The system of claim 18, further comprising:
one or more pressure sensor arrays positioned along the upper brace and the lower brace of the wearable sensor system that are collectively operable for measuring an assistive force applied by a practitioner during the gait rehabilitation exercise.

20. A non-transitory computer readable medium including instructions executable by a processor to:
access a set of observation signals for a time step, the set of observation signals being measured at a body part of a patient during a gait rehabilitation exercise; and
determine, for a time step and by a prediction model implemented at the processor, a predicted attractor state and an assistive torque value for the time step based on the set of observation signals.
